(19) United States
(12) Patent Application Publication
(10) Pub. No.: US 2021/0161634 A1
(43) Pub. Date: Jun. 3, 2021

(54) ULTRASONIC MAMMALIAN TEETH AND GUM TREATMENT SYSTEM AND METHOD

(71) Applicant: Eric HANSEN, Dallas, TX (US)

(72) Inventor: Eric HANSEN, Dallas, TX (US)

(21) Appl. No.: 16/775,090

(22) Filed: Jan. 28, 2020

Related U.S. Application Data
(60) Provisional application No. 62/797,936, filed on Jan. 28, 2019.

(51) Int. Cl. A61C 19/06; A61C 3/03; A61N 7/00

(52) U.S. Cl. CPC A61C 19/06; A61C 3/03; A61N 2007/0043; A61C 2204/002; A61N 2007/0021; A61N 7/00

(57) ABSTRACT

Embodiments of teeth and gum treatment employing ultrasonic transducers are described generally herein. Other embodiments may be described and claimed.

### TECHNICAL FIELD

Various embodiments described herein relate generally to treating teeth and gum pain, including systems, and methods used in treating teeth and gum pain.

### BACKGROUND INFORMATION

It may be desirable to treat teeth and gum pain including pain caused by teething, the present invention provides a system and method for such treatment.

### BRIEF DESCRIPTION OF THE DRAWINGS

FIG. 1A is a simplified side diagram of an ultrasonic mammalian teeth and gum treatment (UMTGT) system according to various embodiments.

FIG. 1B is a simplified, enlarged diagram of the UMTGT system area AA shown in FIG. 1 A with the teeth and gum engaging module operatively removed from the base according to various embodiments.

FIG. 1C is a simplified front diagram of the ultrasonic mammalian teeth and gum treatment (UMTGT) system shown in FIG. 1A according to various embodiments.

FIG. 2A is a simplified side diagram of another ultrasonic mammalian teeth and gum treatment (UMTGT) system according to various embodiments.

FIG. 2B is a simplified, enlarged diagram of the UMTGT system area BB shown in FIG. 2A with the teeth and gum engaging module operatively removed from the base according to various embodiments.

FIG. 2C is a simplified front diagram of the ultrasonic mammalian teeth and gum treatment (UMTGT) system shown in FIG. 2A according to various embodiments.

FIG. 3A-4 are diagrams of signals that may be applied to one or more transducers of a UMTGT system according to various embodiments.

FIG. 5 is a flow diagram illustrating the ultrasonic mammalian teeth and gum treatment (UMTGT) system processing algorithms according to various embodiments.

FIG. 6 is a block diagram of an article according to various embodiments.

### DETAILED DESCRIPTION

Mammals may experience teeth and gum pain due to various factors including teething when young, cavities in a tooth, gum pain due to prosthetics including bridges and dentures. Numbing creams, oil, ice, and chewable appliances may be employed for local treatment. Medicines may be taken to remove pain and inflammation systemically. Each of these treatments have limitations including only treating the symptoms and not the cause of the symptoms. Embodiments of the present invention may alleviate symptoms of teeth and gum pain temporarily and in some cases may treat the underlying cause of the teeth and gum pain.

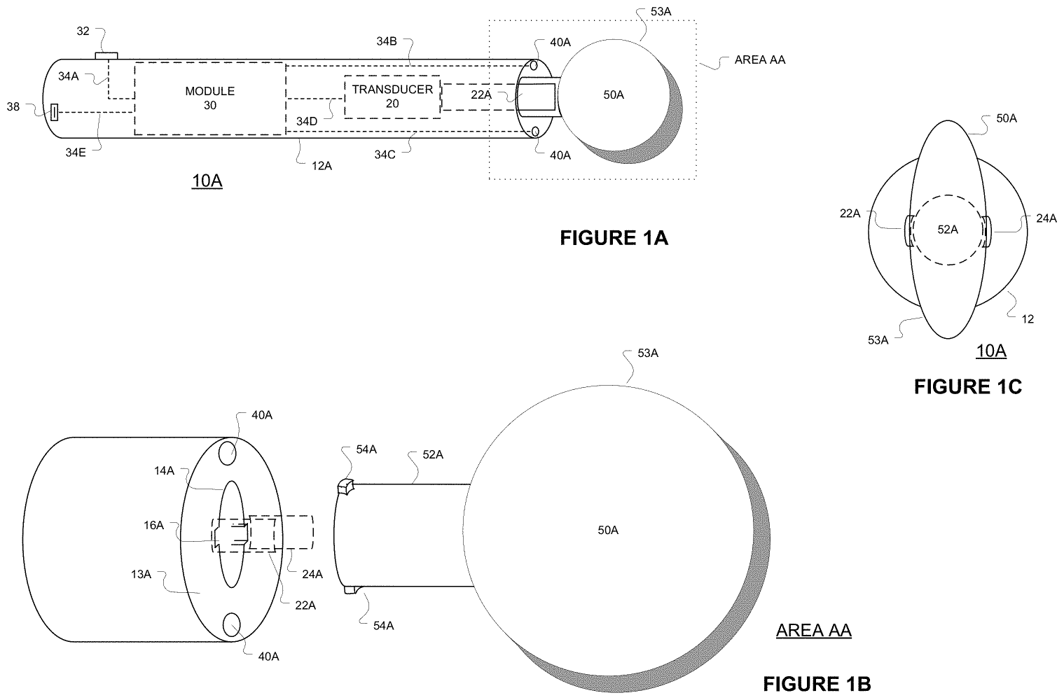

FIG. 1A is a simplified side diagram of an ultrasonic mammalian teeth and gum treatment (UMTGT) system 10A according to various embodiments. As shown in FIG. 1A, the UMTGT system 10A may include a base 12A coupled to a teeth and gum engaging module 50A. FIG. 1B is a simplified, enlarged diagram of the UMTGT system area AA shown in FIG. 1A with the teeth and gum engaging module 50A operatively removed from the base 12A according to various embodiments. FIG. 1C is a simplified front diagram of the ultrasonic mammalian teeth and gum treatment (UMTGT) system 10A shown in FIG. 1A according to various embodiments.

In an embodiment, the teeth and gum engaging (TGE) module 50A may include a treatment head 53A and base 12A engaging extension 52A. The head 53A and extension 52A may be formed of a medical grade, pliable material including silicon, rubber, polymer or combinations thereof. In an embodiment, the TGE module 50A may be permanently attached to the base 12A at end 13A. In an embodiment, shown in FIG. 1B, the TGE module 50A may be removably couplable to the base 12A at end 13A using various mechanisms. In an embodiment, the base 12A end 13A may include an opening 14A having a diameter greater than the diameter of the TGE module 50A extension 52A to enable the secure but removable coupling of the TGE module 50A to the base 12A. The opening 14A may include one or more notches 16A that form channels to engage mating registration tabs MA on the outer diameter of the extension 52A.

As shown in FIG. 1A, the base 12A may include a control module 30, transducer 20, control switch(es) 32, light emitting modules 40A, and module interface 38 therein. The transducer 20 may be coupled to one or more extensions 22A, 24A that are sized to engage to the outer diameter of the TGE module 50A extension 52A when operatively coupled to the base 12A end 13A. In an embodiment, the extensions 22A, 22B may be formed of various hard materials including metals, alloys, ceramics, or hardened polymers. The control module 30 may be electrically coupled to the transducer 20, control switch(es) 32, light emitting modules 40A, and module interface 38 via wires 34D, 34A, 34B, 34C, and 34E, respectively. In an embodiment, the light emitting modules 40A may include one or more light emitting diodes (LED) that output light at various frequencies including in the infrared spectrum of frequencies in an embodiment toward the TGE module 50A.

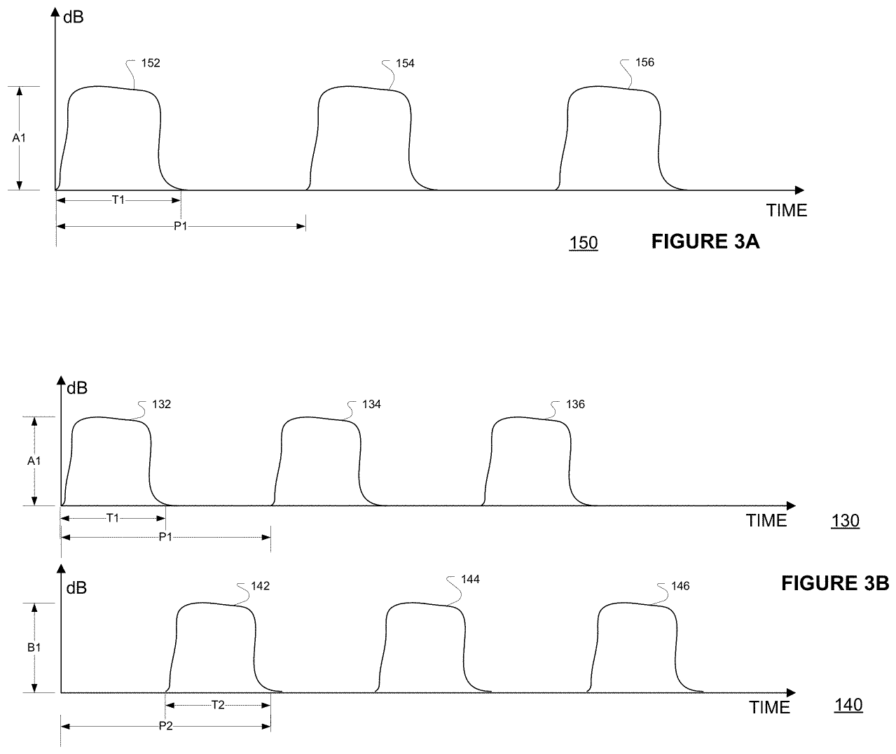

The control module 30 may be directed to provide signals to the transducer 20 to cause the transducer to vibrate the extensions 22A, 24A together or independently, causing the TGE module 50A extension 52A and thus the head 53A to vibrate. In an embodiment, the control module 30 via the transducer may causes the extensions 22A, 24A to vibrate or generate sound waves that cause the extensions 22A, 24A to vibrate at 10,000 to 10,000,000 pulses or twice as many movements per second. The vibration frequency and pattern may be selected or controlled via the control module 30. In an embodiment, the vibration pulses 152, 154, 156 having magnitude A1, duty T1, during period P1 (pulse pattern 150) with a selected vibration frequency as shown in FIG. 3A may be applied to the extensions 22A, 24A. For a User having teeth or gum issues, the vibration of a head 53A at ultrasonic frequencies may temporarily deactivate nerve receptors (numb the pain) for a time interval. For a User having teething issues, the vibration of a head 53A at ultrasonic frequencies may temporarily deactivate nerve receptors (numb the pain) for a time interval and also help speed up the gum cutting process due the vibrations (ultrasonic cutting effect).

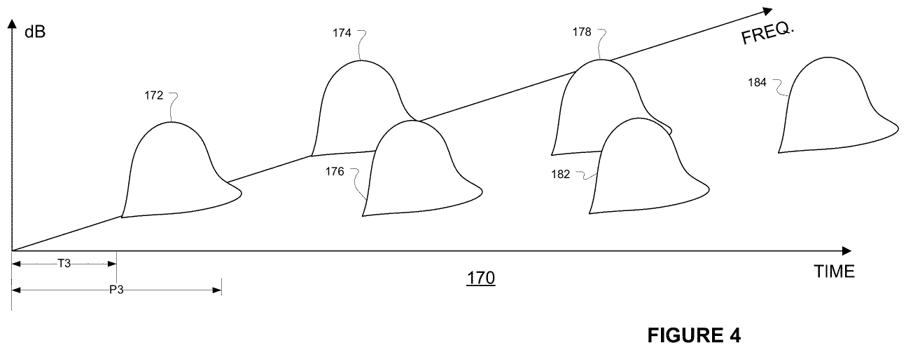

In an embodiment, the vibration pulses 132, 134, 136 having magnitude A1, duty T1, during period P1 (pulse pattern 130) with a selected vibration frequency as shown in FIG. 3B may be applied to the extensions 22A while the vibration pulses 142, 144, 146 having magnitude B1, duty T2, during period P2 (pulse pattern 140) with a selected vibration frequency as shown in FIG. 3B may be applied to the extensions 24A forming offset pulses vibrations to the extensions 22A, 24A and thus to the head 53A. As shown in FIG. 4, pulses 172, 174, 176, 178, 182, 184 of varied vibration frequency having duty T3 over period P3 over time may be applied to the extensions 22A, 24A via the controller 30 and transducer 20. In an embodiment, the control module 30 may cause the light modules 40A to generate light when the transducer is active, during the entire periods or only during the duty cycles.

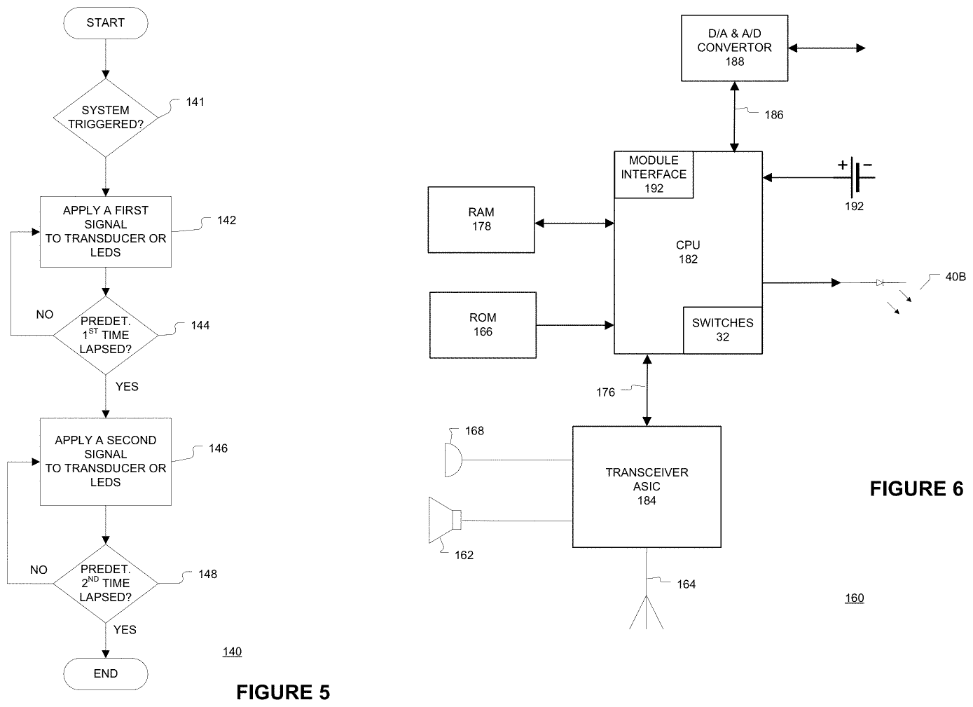

As shown in FIG. 5, the control module 30 may apply a first signal or series of signals to the transducer (activity 142 of algorithm 140) for a predetermined 1^{st} time internal (activity 144) when the UMTGT system 10A is triggered (activity 141). In an embodiment, the module interface 38 may be a power and data interface electrically coupled to the control module 30 enabling a User to program the operation of the control module 30 including vibration patterns to be applied and lighting of LEDs 40A and charge an internal battery (192 of FIG. 6). In an embodiment, the module interface 38 may be a standard interface such as one compliant with a universal serial bus (USB) standard or Apple® standard. In embodiment, the control module 30 may include a transceiver (184-FIG. 6), enabling a User to operate, charge, and control the operation of a UMTGT system 10A. 10B wirelessly using various standards including Bluetooth, zigbee, WiFi, mesh, cellular, and others.

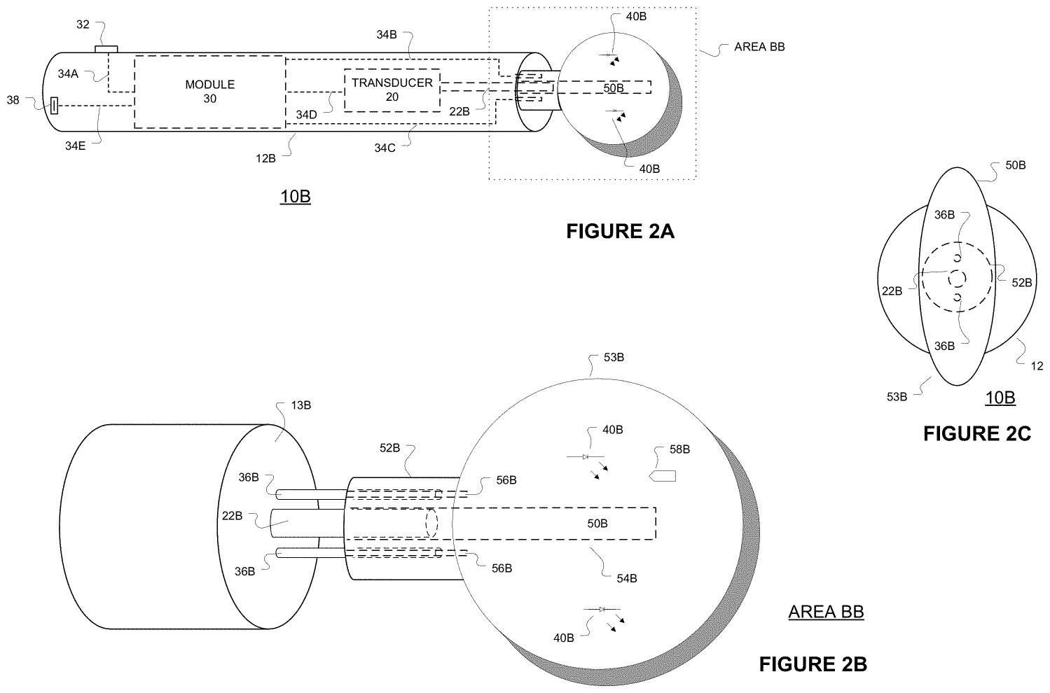

Further, the case 12A may include one or more switches 32 that enable a User to control the operation of the UMTGT system 10A. In an embodiment, the head 53B (shown in FIG. 2B), may include a pressure sensor 58B embedded therein and electrically coupled to the module 30. In an embodiment, the UMTGT system 10 may triggered via the switches 32, the interface 38, detection of pressure in the head 53A, 53B, or via a wireless command. In embodiment, after the 1^{st} time interval has lapsed (activity 144), the module 30 may apply a second signal or groups of signals to the transducer or LEDs 40A for a 2^{nd} time interval (activities 146, 148). FIG. 2A is a simplified side diagram of another ultrasonic mammalian teeth and gum treatment (UMTGT) system 10B according to various embodiments. FIG. 2B is a simplified, enlarged diagram of the UMTGT system 10B area BB shown in FIG. 2A with the TGE module 50B operatively removed from the base 12B according to various embodiments. FIG. 2C is a simplified front diagram of the UMTGT system shown in FIG. 2A according to various embodiments.

As shown in FIGS. 2A-2C, the TGE module 50B may receive an extension 22B and electric connections 36B protruding from the base 12B end 13B. The TGE module 50B extension 52B may include a transducer extension coupler MB and electric signal coupling pins 56B. The transducer extension coupler MB may extend into the head 53B as shown in FIG. 2B. In an embodiment, the extensions 22A, 36B and coupler MB may be formed of various hard materials including metals, alloys, cermanics, or hardened polymers. As also shown in FIG. 2B, the light modules 40B, which may include LEDs may be embedded in the pliable head 53B along with a pressure sensor 58B. It is noted that the head 53A, 53B may have different shapes and sizes a function of the intended User. The head 53A, 53B may be circular from side view (FIG. 1B, 2B) and oval shaped from a top view (FIG. 1C, 2C). It may be sized for human infants' mouths, about 0.5 to 1.5 inches in diameter (from side) and maximum width (from top) of about 0.2 to 0.7 inches in an embodiment. It may be sized or include other sized heads 50A, 50B for human adults' mouths, about 1.0 to 2.0 inches in diameter (from side) and maximum width (from top) of about 0.3 to 1.2 inches in an embodiment.

FIG. 6 is a block diagram of an article 160 according to various embodiments. The article 160 shown in FIG. 6 may be used in various embodiments as elements of the base 12A, 12B and head 50A, 50B including the control module 30. The article 160 may include a central processing unit (CPU) 182, a random access memory (RAM) 178, a read only memory (ROM″) 166, a transceiver application specific integrated circuit (ASIC) 184, a digital to analog (D/A) and analog to digital (A/D) convertor 188, a microphone 168, a speaker 162, and an antenna 164. The CPU 182 may include a module interface 192 and switches 32. In an embodiment, a User may be able to trigger the UMTGT system 10A, 10B via voice commands detected by microphone 168. The module 30 may indicate operational information via the speaker 162 in an embodiment or play various songs having various length to encourage usage of the system 10A, 10B for predetermined periods of time.

The ROM 166 is coupled to the CPU 182 and may store the program instructions to be executed by the CPU 182. The RAM 178 is coupled to the CPU 182 and may store temporary program data, overhead information, and frequency patterns. The microphone 168 and speaker 162 may be incorporated into the base 12A, 12B. Received data may be transmitted to the CPU 182 via a bus 176 where the data may include signals for the transducer 20 and light modules 40A, 40B. The transceiver ASIC 184 may include an instruction set necessary to communicate data, screens, or signals. The ASIC 184 may be coupled to the antenna 164 to communicate wireless messages, pages, and signal information within the signal. When a message is received by the transceiver ASIC 184, its corresponding data may be transferred to the CPU 182 via the serial bus 176. The data can include wireless protocol, overhead information, and operational data to be processed by the module 30 in accordance with the methods described herein.

The D/A and A/D convertor 188 may be coupled to one or more optical modules 40A, 40B to generate a signal to be used to energize one of the optical modules. The D/A and A/D convertor 186 may also be coupled to the transducer 20. Any of the components previously described can be implemented in a number of ways, including embodiments in software. Any of the components previously described can be implemented in a number of ways, including embodiments in software. The modules may include hardware circuitry, single or multi-processor circuits, memory circuits, software program modules and objects, firmware, and combinations thereof, as desired by the architect of the systems 10A, 10B and as appropriate for particular implementations of various embodiments.

They are not intended to serve as a complete description of all the elements and features of apparatus and systems that might make use of the structures described herein.

It may be possible to execute the activities described herein in an order other than the order described. Various activities described with respect to the methods identified herein can be executed in repetitive, serial, or parallel fashion.

A software program may be launched from a computer-readable medium in a computer-based system to execute functions defined in the software program. Various programming languages may be employed to create software programs designed to implement and perform the methods disclosed herein. The programs may be structured in an object-orientated format using an object-oriented language such as Java or C++. Alternatively, the programs may be structured in a procedure-orientated format using a procedural language, such as assembly or C. The software components may communicate using a number of mechanisms well known to those skilled in the art, such as application program interfaces or inter-process communication techniques, including remote procedure calls. The teachings of various embodiments are not limited to any particular programming language or environment.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus, the following claims are hereby incorporated into the Detailed Description, with each claim standing on its own as a separate embodiment.

What is claimed is:

1. A system for treating mammalian teeth and gum pain, including a base including a controllable vibrational transducer; and
a teeth and gum engaging head coupled to the vibrational transducer to receive vibrations
generated by the transducer, the head having two substantially continuous planar surface areas separated by a width less than the heights of the planar surface areas, the head made substantially of a pliable medical grade material and sized to be comfortably insertable into a mammalian month having teeth and gum to be treated.

2. The system for treating mammalian teeth and gum pain of claim 1, wherein the teeth and gum engaging head is coupled to the vibrational transducer via a substantially rigid extension directly coupled to the vibrational transducer.

3. The system for treating mammalian teeth and gum pain of claim 1, wherein the teeth and gum engaging head is removably coupled to the vibrational transducer via a substantially rigid extension directly coupled to the vibrational transducer and indirectly couplable to the teeth and gum engaging head.

4. The system for treating mammalian teeth and gum pain of claim 1, wherein the base includes a proximal end and distal end and a longitudinal axis therebetween, and the teeth and gum engaging head includes an extension extending from the two substantially continuous planar surface areas, the extension configured to be removably couplable to the base distal end.

5. The system for treating mammalian teeth and gum pain of claim 4, further including a substantially rigid extension directly coupled to the vibrational transducer and indirectly couplable to the teeth and gum engaging head extension.

6. The system for treating mammalian teeth and gum pain of claim 1, further including a control module electrically coupled to the transducer and a pressure sensor in the teeth and gum engaging head, the control module providing signals to the transducer to cause the transducer to vibrate for predetermined intervals of time when pressure is detected by the pressure sensor.

7. The system for treating mammalian teeth and gum pain of claim 6, the control module providing signals to the transducer to cause the transducer to vibrate at 10,000 to 10,000,000 pulses movements per second when pressure is detected by the pressure sensor.

8. The system for treating mammalian teeth and gum pain of claim 1, further including a control module electrically coupled to the transducer and a user interface coupled to the control module to enable a user to trigger the operation of the transducer for a predetermined period of time.

9. The system for treating mammalian teeth and gum pain of claim 1, further including a control module electrically coupled to the transducer and a user interface coupled to the control module to enable a user to determine the vibration rate and time of the transducer.

10. The system for treating mammalian teeth and gum pain of claim 1, further including a control module electrically coupled to the transducer and a user interface coupled to the control module to enable a user to determine one of plurality of vibration patterns to be applied to the transducer for a plurality of predetermined time intervals.

11. A method of treating mammalian teeth and gum pain, including employing a vibration system to teeth and gum to be treated, the system including:
a base including a controllable vibrational transducer; and
a teeth and gum engaging head coupled to the vibrational transducer to receive vibrations generated by the transducer, the head having two substantially continuous planar surface areas separated by a width less than the heights of the planar surface areas, the head made substantially of a pliable medical grade material and sized to be comfortably insertable into a mammalian month to engage the teeth and gum to be treated.

12. The method of treating mammalian teeth and gum pain of claim 11, wherein the teeth and gum engaging head is coupled to the vibrational transducer via a substantially rigid extension directly coupled to the vibrational transducer.

13. The method of treating mammalian teeth and gum pain of claim 11, wherein the teeth and gum engaging head is removably coupled to the vibrational transducer via a substantially rigid extension directly coupled to the vibrational transducer and indirectly couplable to the teeth and gum engaging head.

14. The method of treating mammalian teeth and gum pain of claim 11, wherein the base includes a proximal end and distal end and a longitudinal axis therebetween, and the teeth and gum engaging head includes an extension extending from the two substantially continuous planar surface areas, the extension configured to be removably couplable to the base distal end.

15. The method of treating mammalian teeth and gum pain of claim 14, the system further including a substantially rigid extension directly coupled to the vibrational transducer and indirectly couplable to the teeth and gum engaging head extension.

16. The method of treating mammalian teeth and gum pain of claim 11, the system further including a control module electrically coupled to the transducer and a pressure sensor in the teeth and gum engaging head and further including directing the control module to provide signals to the transducer to cause the transducer to vibrate for predetermined intervals of time when pressure is detected by the pressure sensor.

17. The method of treating mammalian teeth and gum pain of claim 16, further including directing the control module to provide signals to the transducer to cause the transducer to vibrate at 10,000 to 10,000,000 pulses movements per second when pressure is detected by the pressure sensor.

18. The method of treating mammalian teeth and gum pain of claim 11, the system further including a control module electrically coupled to the transducer and a user interface coupled to the control module and further including enabling a user to trigger the operation of the transducer for a predetermined period of time.

19. The method of treating mammalian teeth and gum pain of claim 11, the system further including a control module electrically coupled to the transducer and a user interface coupled to the control module and further including enabling a user to determine the vibration rate and time of the transducer.

20. The method of treating mammalian teeth and gum pain of claim 11, the system further including a control module electrically coupled to the transducer and a user interface coupled to the control module and further including enabling a user to determine one of plurality of vibration patterns to be applied to the transducer for a plurality of predetermined time intervals.
